(19) United States
(12) Patent Application Publication
(10) Pub. No.: US 2004/0044272 A1
(43) Pub. Date: Mar. 4, 2004

(54) Personal condition management system

(76) Inventors: Piet Moerman, St.Martens-Latem (BE); Jerry McAleer, Oxon (GB); Robert Oringer, Quebec (CA)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/363,143

(22) PCT Filed: Feb. 8, 2002
(86) PCT No.: PCT/GB02/00548

(51) Int. Cl. A61B 5/00

(52) U.S. Cl. 600/300

(57) ABSTRACT

A personal condition management system comprising an input device, an advisory facility and an output device, wherein: said input device is adapted to communicate historical, current and/or prospective condition-relevant data to said advisory facility; said advisory facility is adapted to receive and store historical, current and/or prospective condition-relevant data from said input device and process said condition-relevant data to generate a treatment recommendation; and said output device is adapted to receive the treatment recommendation from the advisory facility and communicate receipt of said treatment recommendation to a

### FIELD OF THE INVENTION

The present invention relates to a personal condition management system. More specifically the present invention relates to an “intelligent” glucose monitoring system for management of diabetes. However, the system of the invention may also be used for the management of patients with blood clotting disorders, for instance those being treated with anticoagulant therapy or haemophiliacs being treated with Factor VIII.

### BACKGROUND OF THE INVENTION

It has long been recognized that, for people with diabetes, frequent measurement and monitoring of blood glucose can significantly enhance quality of life and reduce occurrence of diabetic complications. To this end, manufacturers of blood glucose monitoring systems have, over the years, invested much effort into designing systems which reduce the barriers to patient self testing, notably inconvenience and pain. However, the typical blood glucose monitoring system is essentially a meter which provides the patient with only a simple measurement of blood glucose level in response to a blood measurement.

In reality, glycaemic control is a highly complex feedback system depending on stress, exercise, food intake, insulin, glucagon, adrenaline and other hormones. Glucose concentration is the result of the interplay of these factors. Two major interventions can be used by the diabetic: food intake (quantity and composition) and insulin administration, of particular quantities and compositions. It is therefore highly desirable to provide information to the patient on the amount of insulin to be injected or food to be consumed rather than a simple glucose level measurement in order effectively to manage diabetes.

In a conventional diabetes monitoring system, a blood sample is obtained from a patient by a lancing device and placed on a detection strip. The strip is subsequently introduced into a test device or meter in a controlled fashion. The meter measures the glucose level in the blood sample. Responsive to the blood glucose reading and prospective food intake or exercise, the patient or a health care practitioner (HCP) administers an appropriate amount of a medicament, such as insulin or glucagon, or the patient ingests a source of glucose, such as a glucose pill or a source of carbohydrate, that is correlated with the blood glucose reading of the meter to control the glucose level of the patient.

In some available meters, there is a facility for entering into a store condition-relevant data, such as amount of medication, amount of glucose ingested, meals eaten or exercise taken. However, such meters are effectively little more than electronic logbooks and do not provide any real-time useful condition feedback or provide intelligent treatment recommendations. Further, data entry into these meters can be a time consuming and complex affair, causing patients to neglect to input relevant data and so defeating the object of the meter. It is possible periodically to download the inputted data, together with glucose measurement data, usually at the site of a HCP. Such periodic downloading normally provides the basis for a therapy review. One limitation of this approach, however, is that such periodic reviews provide condition state management which is much “coarser” than ideal.

### SUMMARY OF THE INVENTION

The present invention provides a personal condition management system comprising an input device, an advisory facility and an output device, wherein:

said input device is adapted to communicate historical, current and/or prospective condition-relevant data to said advisory facility;

said advisory facility is adapted to receive and store said historical, current and/or prospective condition-relevant data from said input device and process all said condition-relevant data to generate a treatment recommendation; and

said output device is adapted to receive the treatment recommendation from the advisory facility and communicate receipt of said treatment recommendation-to a patient

Preferably, the advisory facility will process the condition-relevant data to provide a continuous estimate of the level of a condition-relevant analyte, such as blood glucose, and generate the treatment recommendation when the analyte level is predicted to fall outside preset limits.

The present invention allows for extremely comprehensive and facile input of condition relevant data. The present invention also solves the “coarse” management problem referred to above by providing immediate data analysis within the system's advisory facility. The present invention provides an intelligent condition management system for providing recommendations for treating the condition. The management system of the present invention facilitates the management of a condition, such as diabetes, by providing appropriate treatment recommendations depending on a number of condition-relevant input parameters.

The condition-relevant data will generally include measurements of a fluid analyte, such as blood glucose. These measurements may be input by the patient. Preferably, however, the input device for inputting these measurements is a meter which makes the measurements. The output from such a meter may be automatically inputted to the advisory facility. The output of the meter may be communicated to the advisory facility by wireless transmission, for instance by use of infrared or radio frequencies, or by “docking” a communications port on the meter with a communications port on the advisory facility. Where the meter can be located on the body, such as a glucose meter using infrared spectroscopy to measure glucose levels continuously, and the advisory facility can also be located on the body, the output of the meter may be communicated to the system using the body to conduct the signals. Preferably, the advisory facility and the meter are part of a unitary instrument and the output from the meter is communicated to the advisory facility by hard wiring.

The condition-relevant data will also generally include measurements of medication taken by the patient. Again, these measurements may be input by the patient of the system. Preferably, however, the input device for inputting these measurements is a dosing device which is used to administer the medicament. This could be a syringe, an insulin pen or a pump located within the body. The output from such a dosing device may be automatically inputted to the advisory facility. The output of the dosing device may be communicated to the advisory facility by wireless transmission, for instance by use of infrared or radio frequencies, or preferably by “docking” a communications port on the dosing device with a communications port on the advisory facility. Alternatively, the advisory facility and the dosing device may be part of a unitary instrument and the output from the dosing device is communicated to the advisory facility by hard-wiring. Where the dosing device can be located on the body, at least in use, such as an insulin pen or a pump, and the advisory facility can also be located on the body, the output of the dosing device may be communicated to the advisory facility using the body to conduct the signals.

The condition-relevant data may also include other data which can be generated by measurements carried out on the body. For instance, in diabetics and people suffering from high blood pressure, stress is a very important factor. Therefore, it would be useful to input data regarding blood pressure and/or heart rate, and possibly also electrocardiogram data. These data can all be measured instrumentally and the output from these instruments can be input into the advisory facility. The input may be made by any of the methods mentioned above. Preferably, the data are communicated to the system by wireless communication or, when the system and the instruments are located on the body, using the body to conduct the signals.

The condition-relevant data may also include those which need to be input by the patient These include levels of stress, amount of food ingested or to be ingested and amount of exercise taken or to be taken. In order to make the system as patient friendly as possible, the device used to input these data should be easy to use.

Thus, the input device may comprise a display screen controlled by one or more buttons which allow the patient to scroll through a menu and select items to be input For instance, a menu for a diabetic may have a first screen which allows the patient to select “Insulin”, “Glucose”, “Carbohydrate”, “Meal”, “Wine” “Exercise” or “Stress”. Once one of the menu items is selected, a new menu appears giving either a qualitative, such as “low”, “medium” or “high”, or a quantitative measure of the selected item. Once one of these menu items is selected, a new menu may appear giving the patient a selection of times, such as “2 hours ago”, 1 hour ago”, “now”, “1 hour later” or “2 hours later”.

Alternatively, the menus may be navigated through using a responsive screen and a stylus, in a similar manner to that used with Palm® or similar hand held computers.

Preferably, the menus are accessed using voice recognition software in the input device.

It is not necessary to use a menu-driven input device. It is also possible to use a keyboard or keypad or, preferably, voice recognition software to enable the direct input of the condition-relevant data. For instance, the voice recognition software could be trained to recognise such phrases as “one dose of insulin”, “50 grams of carbohydrate”, “1 mile jog” or “heavy breakfast” and then input to the advisory facility appropriate condition-relevant data.

The advisory facility will include means for receiving the input data and means for outputting signals to the output device. It will also be able to process the data to generate the treatment recommendation. Preferably, the advisory facility will comprise a microcomputer having memory units, a processor and data busses as are well known in the art. No detailed description of such microcomputers is provided he-re as their construction forms no part of the present invention.

The inputted data is processed according to an algorithm. The algorithm will be based on studies of the effects of the various condition-relevant data on populations of people who have the condition. It should also take into account, as far as possible, universal variables such as seasonal variations and sleeping patterns. However, such population-related algorithms will not necessarily be ideal for each individual patient. Therefore, preferably, the algorithm is able to “learn” about an individual patient. This may be achieved either by making alterations to the algorithm following a study of the condition-relevant data collected by the system over a period of time or by designing the algorithm so that it can “learn” as it receives the data. Thus, the algorithm may show “artificial intelligence”.

In particular, it is preferred that the algorithm is able to learn the life patterns of the individual patient and prompt him to provide information in accordance with his life pattern. For instance, for a diabetic, it is important to record food intake. If the algorithm has learned that the patient regularly eats breakfast at 7:30 in the morning, and by 7:45 no data have been input, then the advisory facility should generate a prompt asking the patient to enter data relating to his breakfast. If the advisory facility has recorded that the patient's breakfast involves consuming a standard amount of carbohydrate, the prompt may ask the patient to confirm that the standard amount was consumed. In this way, the patient will find it easier to keep the data in the advisory facility complete.

Preferably, the advisory facility is reprogrammable so that general changes in the management of the condition can be incorporated into its algorithm. For instance, the changes in the algorithm may be affected by “Case Base Reasoning”, where data from a population of people with the condition and advice from professionals who manage the condition in a number of patients is used to modify recommended treatments for the condition.

The algorithm is designed so that it produces a treatment recommendation between data inputs initiated by the patient, such as the input of a measurement initiated by the patient. The system may also be designed to produce a treatment recommendation at the time of patient-initiated data input. However, at the time of such data input, the patient is normally aware of his condition and so will not need a treatment recommendation. The patient will not be so aware of his condition between patient-initiated data inputs and so the provision of treatment recommendations between patient-initiated inputs will be more helpful for the patient.

The treatment recommendation may be very simple, such as “Carry out a test” or “Consult doctor”. If the treatment recommendation is not urgent, merely prompting the patient to carry out a test may lead the patient to take the appropriate action. If the treatment recommendation is more urgent, such as when a diabetic requires an injection of glucagon, a “Consult doctor” message should be sufficient to cause the patient to take appropriate action.

However, the treatment recommendation may, if desired, be more detailed. For instance, for a diabetic, the patient may require insulin to reduce glucose levels or glucose if his glucose level is too low. In some situations, it is necessary to provide the glucose in a readily available format, such as in the form of a glucose tablet or a candy bar. In other situations, the glucose should be provided over a longer time course, such as by the ingestion of a complex carbohydrate source, for instance pasta. Thus, for a diabetic, the treatment recommendation may be “Take insulin?”, “Take glucose tablet?”, “Eat candy bar?” or “Eat carbohydrate?”. As the system learns about the individual patient, it will become possible to provide more specific treatment recommendations, such as “Take 1 dose of insulin”, “Take 5 g glucose tablet”, “Eat 1 candy bar” or “Eat 50 g pasta”.

The output device may produce a very simple audible, tactile or visual alert signal, such as a buzzer or a vibrating device or a flashing or moving display, such as those that are used on mobile telephones. The output device will alert the patient to the fact that the advisory facility has determined that a treatment recommendation has been produced.

However, the output device may also be, or additionally include, a more sophisticated device, such as a mobile telephone which is programmed to telephone or send a text message to one or more particular telephone numbers. This will be advantageous, for instance, for children suffering from the condition, in that it will be possible for both the child and one or both of his or her parents, or his or her HCP, to receive the communication from the advisory facility.

Preferably, the output device also displays or makes audible the treatment recommendation. Where the input device uses a screen for input, the same screen can be used to display the treatment recommendation. Where the input device uses voice recognition software, its microphone may also be used as a loudspeaker to provide the treatment recommendation audibly. Where the output device is or includes a mobile telephone, the treatment recommendation may be sent as a text message or a voice mail.

The output device may include more than one means for alerting the patient to the fact that the advisory facility has produced a treatment recommendation. Thus, the output device may provide any combination of audible, tactile and visible alerts.

Preferably, the system also includes means for interfacing the system with other systems. For instance, the system may be provided with a communications port for docking with a computer system, such as the computer system of the HCP treating the condition. This will allow the data stored in the system to be downloaded to the physician's system so that he can fully monitor the patient's condition. Alternatively, the communications port may be able to dock with a general purpose computer system, for instance to enable the stored data to be transmitted to the HCP or a general data base via the internet. Instead of a communications port, the system may be provided with a wireless communication facility, such as an infrared or radio frequency transmitter, whereby the data can be transferred remotely.

Preferably, the system is set up so that upgrades for the algorithm and/or messages from patient's HCP can be downloaded to the system via the communications port or the wireless communication facility.

The present invention thus provides a personal condition management system that allows extremely facile input of condition-relevant data and, in addition, employs an advisory facility to provide advisory therapy recommendations. The system can employ input data about a fluid analyte, such as glucose, and processes the information with the advisory facility. The condition-relevant data input can be effected by “plain language” voice activation with key word recognition enabled within the management system. Conventional manual input techniques can also be employed by utilizing a keypad or keyboard. The data can alternatively be acquired through telecommunications (in case the advisory facility is physically separated from the testing system).

In the case of diabetes management, the advisory facility may provide, indirectly or directly, recommended dosages for long acting and/or fast acting insulin, as well as recommended carbohydrate intake. The advisory facility includes an algorithm running within the advisory facility that uses, as input, historical responses to a specific glucose/insulin/carbohydrate regimen, as well as prospective inputs such as the anticipated carbohydrate load provided by a subsequent meal, or the anticipated duration and intensity of an exercise session.

The treatment recommendation may also be a prospective recommendation based on the patient taking a specific amount of medication. The prospective recommendation may be provided in text, numerical and/or graphical form, and will advise the patient on what might happen if he takes a specific amount of insulin. This allows the advice to be a representation of prospective glucose level behaviour depending on entered values of the variables. The patient can vary the variables so that the prospective glucose values fall within a safe therapeutic range. A tighter and more effective glycaemic control is achieved by making frequent small adjustments to insulin therapy or glucose or carbohydrate intake, based upon historical patient responses.

It will be appreciated that the system of the present invention will be of great use as an educational tool for the patient. It will be possible for the patient to see what effects his life style will have on his condition and will therefore be able more actively to manage his condition and to recognise when he needs to take prophylactic action to maintain his condition under control. This should reduce the need for the patient to directly consult his HCP.

The present invention is now described, by way of example only, with reference to the accompanying drawings.

### BRIEF DESCRIPTION OF THE DRAWINGS

FIG. 1 is a schematic block diagram of a personal condition management system according to the teachings of the present invention.

FIG. 2 is a schematic block diagram of an alternative embodiment of a personal condition management system according to the teachings of the present invention.

FIG. 3 is a schematic flowchart diagram illustrating a method used in the system of the present invention.

FIG. 4 illustrates use of the personal condition management system of FIG. 2 in operation with an insulin delivery device for providing insulin dosage readings to the system.

### DETAILED DESCRIPTION OF THE PREFERRED EMBODIMENTS

The illustrated monitoring system 10 of the present invention receives input data, such as data of a diabetes related event, and employs an advisory facility 16 to generate a treatment recommendation 23 and/or 25 that can be displayed on the display 18 or transferred to the HCP 24 or a health care organization (HCO) 22.

Thus, for example, the advisory facility 16 may be programmed for a diabetic patient to recognize a particular set of factors, e.g. measured glucose level obtained from meter 20, amount of exercise and amount and type of food consumed, insulin dosage and so on, compare the factors to historical, predicted or prospective data and effects of therapy, and calculate an advisory or recommended course of action. Such action may be, for example, to administer a specific fast-acting/slow-acting insulin cocktail, to take carbohydrate or perhaps even to seek medical advice immediately. This advice or recommendation may be transmitted by audio, visual or tactile means, such as a warning beeper or through the system display 18, and may be accompanied by output communications.

Alternatively, the recommendation can be a simulation of what might happen in the near future under the current status of the variables and the selected amount of insulin. The prospective information is based on basic feedback mechanisms which are customised to the patient's own biological makeup or recent historical trends. In order to ensure complete patient safety, the system 10 can employ predetermined limits or ranges on the treatment regimen. The limits can be pre-programmed in the system or provided by the HCP or HCO and coded into the system. Similarly, such limits may be adjusted periodically by the HCP on the occasions of the patient's regular check-up, during which the HCP reviews the historical data stored in the memory of the advisory facility 16 of the monitoring system 10.

The algorithm, stored in the advisory facility 16, which processes the input data and formulates treatment/monitoring advice or recommendations can be defined by the HCP. Suitable algorithms and parameters can be devised based on algorithms which are well known in the art and available to HCPs in the field. The system 10 can be programmed with the algorithm directly or “uploaded” with the algorithm through a wired or wireless connection with the HCP 24. The HCP can, if he wishes, have a profound influence on the data processing algorithm. For example, he may want to set the insulin sensitivity for that specific patient himself or may change the carbohydrate consumption in function of the Lean-Body-Mass of the patient.

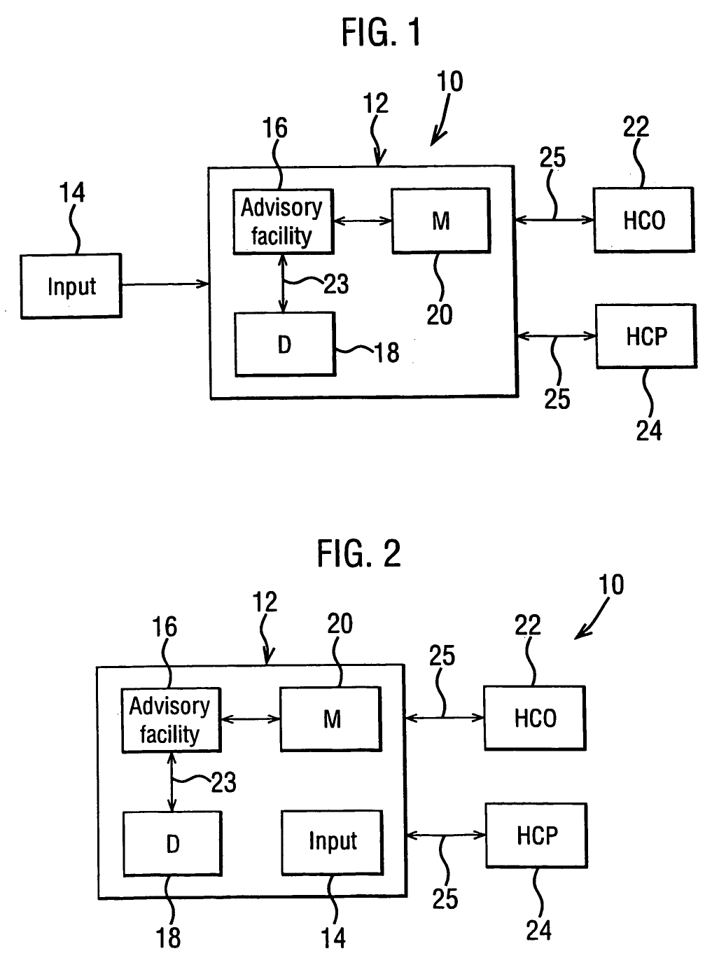

FIG. 1 illustrates a schematic block diagram of a personal condition management system according to the teachings of the present invention. The illustrated system 10 includes an integrated instrument 12 that is coupled to an input device 14 and one or more output facilities 22 and 24. The illustrated instrument 12 includes an advisory facility 16 that is coupled in single or bi-directional communication with a display element 18 and a meter 20.

The input device 14 is intended to provide for the input of data into the advisory facility 16. As shown in FIG. 1, the input device 14 can be coupled to the instrument 12 by any conventional hard wired connection or through a wireless in or radio frequency connection or through use of the patient's body as a transmission medium. The patient may enter condition-relevant data, such as information on insulin dosage, diet, exercise and stress levels. The condition-relevant data can also be provided from a blood sample collected using a lancing device and measured using a non-integral meter, such as a glucose meter. The input device 14 is intended to provide for the input of unmeasured data into the instrument 12.

The term “meter” as used herein is intended to include any suitable electronic device that is capable of generating fluid analyte or constituent related data directly from the patient or from another device. Examples of suitable meters can include glucose meters, semi-continuous or continuous glucose monitors, oximeters or other non-invasive measuring instruments, and the like.

According to one practice, the condition can be diabetes, and the fluid analyte can be a blood constituent such as glucose and the meter 20 is a glucose meter. The data can be measured from a blood sample collected from a lancing device and applied to a test strip located in or to be located in the meter. The meter acts as an input device for providing measured condition-relevant data to the advisory facility 16.

In an alternative embodiment, the separate input device 14 may be dispensed with and the display 18 used as both the display and as the input device. In a further alternative, as shown in FIG. 2, a separate input device 14 may be incorporated into the instrument 12. This may be a keypad or keyboard on the device or a microphone whose output is processed by voice recognition software run by the advisory facility 16.

The advisory facility 16 generates a treatment recommendation for the patient in response to input data that can be displayed on the display 18 or transferred to the HCP 24 or a health care organization (HCO) 22.

The illustrated instrument 12 is a “smart” or intelligent system since it can process or employ selected, contemporaneous predetermined or pre-stored data to generate a treatment recommendation to a patient. For example, the advisory facility 16 enables the instrument 12 to utilize or process historical data regarding an individual's response to certain selected treatment regimens for a condition. At the same time the advisory facility 16 can utilize relevant prospective data, such as the anticipated carbohydrate intake for a meal, or anticipated duration and intensity of an exercise session. When the required data is not available, the system can request that the data be entered through the input device 14 so an advice or recommendation can be formulated.

The historical responses can be stored in a memory in the advisory facility 16, or stored at a remote location, and can be processed by the advisory facility 16 in order to generate a treatment recommendation. The standard data that can be employed by the advisory facility 16 include historical responses to regimens, selected glucose information, both recent and historical, the efficacy of previous treatment regimen, as well as certain selected environmental, personal or phenotypic data, including stress, amount of exercise, type and/or amount of food, amounts and frequency of insulin administration, the amounts and frequency of glucagon administration and the patient's insulin sensitivity. Additionally, the system may require that selected prospective data be inputted to the system, such as the time of the next meal and exercise session. The system may also produce prompts based on the life pattern of the patient, asking for particular data inputs related to the patient's condition or for confirmation that an expected event has occurred.

The foregoing data can be stored in the advisory facility 16. The illustrated advisory facility 16 can process the information to determine an appropriate medicament dosage, such as the amount of insulin that a patient can employ to treat the condition, such as diabetes. The output of the advisory facility 16 can include how much insulin is necessary, as well as certain selected insulin mixtures or cocktails, whether carbohydrates are needed, as well as advising the subject whether or not medical attention is necessary or can be merely a message to the effect that the patient should carry out a test, for instance of glucose level.

The illustrated instrument 12 can be coupled to an HCO 22 and/or an HCP 24 through a hardwired or wireless connection.

The treatment recommendation 23 and/or 25 may be in the form of simple advice or calculated carbohydrate or insulin injection requirements. Such calculations may include timing and dosage for different insulin types. A simple insulin dosage algorithm includes a target glucose value and a prescribed “normal” pre-prandial (i.e. pre-meal) insulin dose. The recommended dose can be incremented or decremented from the “normal” dose in proportion to the deviation of the reading from the target value. The constant of proportionality and the maximum and minimum dosages can be parameters programmed by the HCP 24.

By allowing the HCP to adjust the instrument advised treatment regimen limits and/or the algorithm for data processing and advice, as described above, the HCP can act as a virtual “personal trainer” for each of his patients. Thus, by way of example, a patient may take a blood sample with a conventional lancing device, apply the blood sample to a test strip, insert the strip in the meter 20 and obtain a measurement of his glucose level, which turns out to be 220 mg/dl. The patient may also enter selected data with the input device 14, such as the key words: Stress, Moderate; Squash +2 hours. By cross checking the recent glucose data and insulin treatment with historical data, the advisory facility 16 can calculate and recommend the most effective treatment, for example, 12 Units slow acting insulin, 150 g carbs. Such recommendation 23 may be presented as a visual display on the display unit 18 and/or as audio using voice synthesis software. The advice can also take the form of a prospective graphing of what might be the glucose levels in the near future. The patient can then alter the variables under his control (amount of insulin to be injected, the duration and intensity of exercise, taking an extra snack, the next meal composition or amount) and make sure that his glucose levels fall within the safe minimum and maximum levels.

The system 10 of the present invention includes a data input for entering relevant patient data. According to an illustrative embodiment, the glucose monitoring system of the present invention includes voice recognition software and hardware configured to recognise certain key words such as “carbohydrates”, “alcohol”. “fats”, “protein”. “exercise”, “light”, “‘medium”, “heavy”, “Insulin”, ‘fast”, “slow” and numerical values such as “5”, “10”, “20”. With such a system, the patient can simply navigate a list of predetermined menu-options and enter relevant data in a number of ways, such as by speaking into the instrument in much the same way as a mobile phone with voice commanding.

A significant advantage of the system 10 of the present invention is that it requires far less time to enter data. Glucose values can be stored automatically with date and time when a measurement is made, as is routine for current glucose monitoring systems. Alternatively, data can be entered through a keypad, a wireless transmission medium or using the patient's body as a transmission medium. The system can then determine an appropriate advisory or treatment regimen for the selected or specified condition. Automatic data acquisition for insulin intakes facilitates the ease of use by limiting the burden of manual data inputs. This feature works with a hard-wired or wireless link to an electronic insulin pen or insulin pump.

According to an alternate embodiment, the system 10 of the present invention adapts to the well defined habits and recognizes the life-style of individual patients. For example, the patient may have favourite foods, drinks, sports and the like. In this case, it is possible to train the system 10 regarding the meaning of specific key words. For example, the instrument 12 may be trained to interpret the word “Squash” as 600 calories, “Walk” as 200 calories and “Wine” as 1 unit of alcohol. The system may also be able to recognize when, during a normal week, these activities take place. Thus, the system can allow the patient at any point to enter a new key word together with its disease relevant interpretation or simply confirm a suggested input. The advantages of such a system are that data are entered unambiguously, quickly and simply. A patient faced with a new lifestyle event simply has to train the instrument, one time, how the event should be interpreted and, from there on, data entry is facile.

The advisory facility 16 may also include provision of “second order” parameters to provide for deviations from the normal treatment For example, the advised insulin dose can be reduced by 10% if intense exercise is anticipated. The advisory facility 16 can also modify the calculated therapeutic recommendations to account for historical patient specific data. Whilst all patients respond broadly the same way to food and insulin, every patient has an individual response profile (i.e. insulin sensitivity). Thus, by reviewing historical data and comparing results obtained under similar circumstances, the instrument 12 can perform finer, patient specific treatment adjustments.

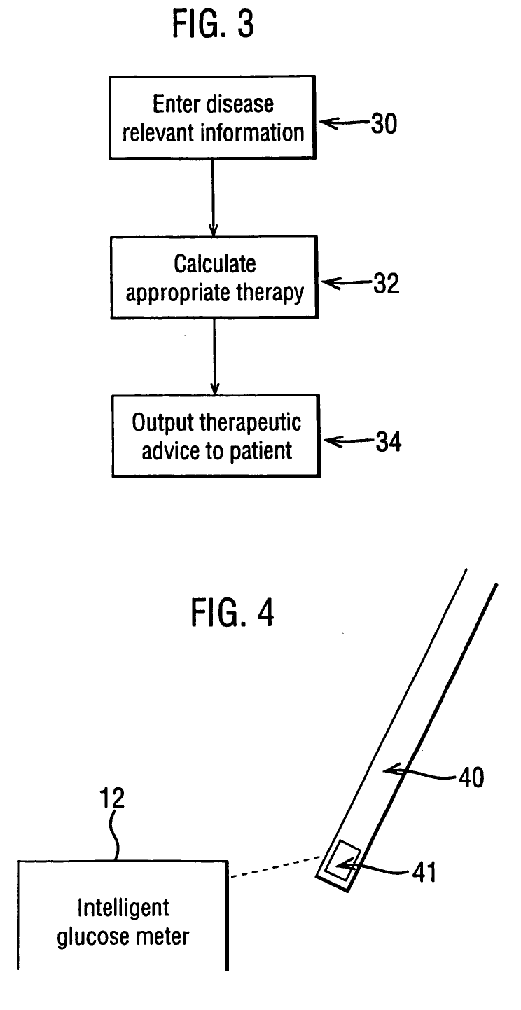

FIG. 3 is a schematic flowchart diagram of the process of managing diabetes which is executed in the illustrative embodiment of the system of the present invention. In step 30, the patient enters condition-relevant information into the instrument 12 with the input device 14. The step of entering disease relevant information includes recording a current glucose reading from the meter 20. In step 32, the advisory facility 16 calculates an appropriate treatment regimen in response to selected input information and for historical data. In step 34, the advisory facility outputs therapeutic advice to the patient.

The features described above can be readily expanded to make the monitoring system 10 even more useful and patient-friendly. For example, the above description assumes the patient enters all relevant data including insulin therapy. However, as shown in FIG. 4, the system 10 can be readily adapted to provide automatic two-way communication with the patient's insulin delivery device 40. If the patient uses an insulin pen, a transmitter on the pen can broadcast the time and details of each insulin injection. Alternatively, the insulin pen can be provided with a communications port for docking with a complementary docking port in the instrument 12. On docking, the pen downloads to the system information on doses given by the pen. The data are received by the system 10 and automatically logged in the database for future reference. Such an approach not only removes one more action for the patient, but also provides a level of security since the data logged reflects the amount and type actually injected.

The same approach also applies to patients on insulin pumps and patients using semi-continuous or continuous glucose monitors. In the latter case, because of the high density of data, the data may be more efficiently stored as trends rather than discrete values. For example, if measurements are recorded every minute and are stable between 85 and 95 mg/dl over a 45 minute period, it is more effective to store the mean value and time limits (3 data points) than each individual reading and corresponding time (90 data points). Similarly a linear trend would be stored as start and end values.

A further expansion of the system features allows for the instrument to communicate with other devices and individuals, for example the mobile phone of a parent, a designated HCP or even the emergency services, to provide an added level of security and care for the patient or to allow for human intervention by a care giver in case the algorithm is “stuck”. This feature may add greatly to the utility as well as the safety of the advice.

According to an alternative embodiment of the invention, the system may comprise a glucose-measuring module for a mobile phone, palm-top device with integrated modern or other communication device. The benefit of this approach is that much of the complex software and hardware is incorporated in the “parent” device, reducing the cost and complexity of the device. For example, a phone can include a facility to recognise speech and convert words to a data stream, together with a sophisticated display, keypad and other useful patient-interface attributes.

It will be recognized that whilst the system described above has been considered specifically from the point of view of the management of diabetes, the principles involved are equally applicable to other self-medicated conditions. Thus for example, patients on anti-clotting therapy may use a similar system to balance Warfarin medication and measured clotting time. Accordingly, the preferred embodiments of the invention as set forth above are intended to be illustrative and not limiting. Various changes can be made without departing from the spirit and scope of the invention.

These examples are meant to be illustrative and not limiting. The present invention has been described by way of example and modifications and variations of the exemplary embodiments will suggest themselves to skilled artisans in this field without departing from the spirit of the invention. Features and characteristics of the above-described embodiments may be used in combination. The preferred embodiments are merely illustrative and should not be considered restrictive in any way. The scope of the invention is to be measured by the appended claims, rather then the preceding description and all variations and equivalents that fall within the range of the claims are intended to be embraced therein.

1. A personal condition management system comprising an input device, an advisory facility and an output device, wherein:
said input device is adapted to communicate historical, current and/or prospective condition-relevant data to said advisory facility;
said advisory facility is adapted to receive and store historical, current and/or prospective condition-relevant data from said input device and process said condition-relevant data to generate a treatment recommendation; and
said output device is adapted to receive the treatment recommendation from the advisory facility and communicate receipt of said treatment recommendation to a patient

2. The system of claim 1 wherein the advisory facility processes the condition-relevant data to provide a continuous estimate of the level of a condition-relevant analyte and generate a treatment recommendation when the analyte level is predicted to fall outside preset limits.

3. The system of claim 1 or claim 2, wherein the input device includes a meter in communication with the advisory facility for communicating to the advisory facility measurements of a condition-relevant fluid analyte.

4. The system of claim 3, wherein the output from the meter is automatically inputted to the advisory facility.

5. The system of claim 3, wherein the output of the meter is communicated to the advisory facility by wireless transmission.

6. The system of claim 3, wherein the output of the meter is communicated to the advisory facility by docking a communications port on the meter with a communications port on the advisory facility.

7. The system of claim 3, wherein the advisory facility and the meter are part of a unitary instrument and the output from the meter is communicated to the advisory facility by hardwiring.

8. The system of any one of claims 2 to 7, wherein the meter is a glucose meter.

9. The system of any one of claims 1 to 8, wherein the input device includes a dosing device in communication with the advisory facility for communicating to the advisory facility measurements of medication taken by a patient of the system.

10. The system of claim 9, wherein the dosing device is adapted to receive dosing instructions from the advisory facility and administer a required dose to the patient.

11. The system of claim 10 or claim 11, wherein the output from the dosing device is automatically inputted to the advisory facility.

12. The system of claim 10 or claim 11, wherein the output of the dosing device is communicated to the advisory facility by wireless transmission.

13. The system of claim 10 or claim 11, wherein the output of the dosing device is communicated to the advisory facility by docking a communications port on the dosing device with a communications port on the advisory facility.

14. The system of any one of claims 1 to 13, wherein the input device includes means for measuring condition-relevant data on the patient's body.

15. The system of claim 14, wherein the measuring means is a blood pressure monitor, a heart rate monitor or an electrocardiogram.

16. The system of any one of claims 1 to 15, wherein the input device includes a display screen controlled by one or more buttons which allow the patient to scroll through a menu and select items to be input.

17. The system of any one of claims 1 to 16, wherein the input device includes a responsive display screen controlled by a stylus.

18. The system of any one of claims 1 to 17, wherein the input device includes voice recognition software.

19. The system of any one of claims 1 to 17, wherein the input device includes a keypad keyboard.

20. The system of any one of claims 1 to 19, wherein the advisory facility comprises a microcomputer which is controlled by an algorithm.

21. The system of claim 20, wherein the algorithm is able to “learn” about an individual patient.

22. The system of claim 20 or claim 21, wherein the algorithm is defined by a health care professional.

23. The system of any one of claims 1 to 22, wherein the advisory facility is reprogrammable.

24. The system of any one of claims 1 to 23, wherein the output device is an audible, tactile or visual device.

25. The system of any one of claims 1 to 24, wherein the output device is, or additionally includes, a mobile telephone which is programmed to telephone or send a text message to one or more particular telephone numbers.

26. The system of any one of claims 1 to 25, wherein the output device displays or makes audible the treatment recommendation.

27. The system of any one of claims 1 to 26, which further includes means for interfacing the system with other systems.

28. The system of claim 27, wherein the interfacing means comprises a communications port for docking with a general purpose computer system or a wireless communication facility.

29. The system of any one of claims 1 to 28, wherein the treatment recommendations take the form of a graphical representation of prospective prediction of what the evolution of glucose levels would be.

30. The system of claim 29, wherein the patient can change some of the variables so to find the safe and therapeutic evolution of glucose levels for the near future.

31. A personal condition management system comprising:
an input device for receiving condition-relevant data; and
an advisory facility for processing the condition-relevant data and generating a treatment recommendation for treating the condition.

32. The system of claim 31, wherein the system is adapted to transfer the treatment recommendation to a health care practitioner.

33. The system of claim 31 or claim 32, wherein the condition is diabetes and the treatment recommendation comprises a recommended insulin dose.

34. The system of any one of claims 31 to 33, wherein the condition is diabetes and the condition-relevant data includes prospective data related to food intake, exercise and insulin dosage.

35. An intelligent glucose meter for meaning and/or predicting glucose levels and managing diabetes in a patient, comprising;
an input unit for receiving diabetes-relevant data for the patient;
an advisory facility for storing said diabetes-relevant data and instructions for generating a treatment recommendation in response to said diabetes-relevant data and for executing said instructions and generating said treatment recommendation; and
an output unit for transferring said treatment recommendation to the patient.

36. The intelligent glucose meter of claim 35, further comprising a communications link for transferring the treatment recommendation to a health care practitioner.

37. A method of managing diabetes in a patent comprising:
providing an intelligent glucose meter for measuring and/or predicting glucose levels and managing diabetes in a patient comprising: an input unit for nerving diabetes-relevant data for the patient; an advisory facility for storing said diabetes-relevant data and instructions for generating a treatment recommendation in response to said diabetes-relevant data and for executing said instructions and generating said treatment recommendation, and an output unit for transferring said treatment recommendation to the patient;
entering diabetes-relevant data through the input facility;
executing the instructions to provide a treatment recommendation in response to the diabetes-relevant data; and
transferring said treatment recommendation to a patient.
